(19) United States
(12) Patent Application Publication
(10) Pub. No.: US 2020/0229715 A1
(43) Pub. Date: Jul. 23, 2020

(54) Image Blood Pressure Measuring Device and Method Thereof

(71) Applicant: FaceHeart Inc., Hsinchu City (TW)

(72) Inventors: Tsuey-Huey Shiue, Hsinchu City (TW); Yu-Fan Fang, Taoyuan City (TW); Po-Wei Huang, Yunlin County (TW); Kuan-Hung Chen, Hsinchu City (TW)

(21) Appl. No.: 16/391,265

(22) Filed: Apr. 22, 2019

(30) Foreign Application Priority Data
Jan. 19, 2019 (TW) 108102106

(51) Int. Cl. A61B 5/021; A61B 5/024; A61B 5/00; G06T 7/00

(52) U.S. Cl. CPC A61B 5/02108; A61B 5/02416; A61B 5/0077; A61B 5/0064; A61B 5/7278; A61B 5/0013; A61B 5/6898; G06T 7/0012

(57) ABSTRACT

A method for evaluating systolic and diastolic blood pressures of a subject for a blood pressure detecting system is disclosed. The blood pressure detecting system includes a processing module and an image capturing module configured to continuously records a hand and a face of a subject to retrieve multiple hand and face images associated with the subject. The method includes, by the processing module, obtaining biological information related to blood pressure of the subject according to the multiple hand and face images retrieved by the image capturing module; and, by the processing module, obtaining prediction results of systolic and diastolic blood pressures of the subject according to the biological information related to blood pressure of the subject.

### BACKGROUND OF THE INVENTION

### 1. Field of the Invention

The present invention relates to an image blood pressure measuring device and method, and more particularly, to an image blood pressure measuring device and method using image pulse wave time difference.

### 2. Description of the Prior Art

In a conventional image blood pressure measuring device, there area front camera and a back camera to simultaneously measure a finger pulse wave signal and a face pulse wave signal of a subject to evaluate a time difference between the finger pulse wave signal and the face pulse wave signal. The subject has to hold the measuring device (e.g., a smart phone) to perform the measurement; however, it is inconvenient for some occasions (e.g., the subject is driving a car). In the prior art, the measuring method is to calculate a finger and face pulse wave peak signal time difference to be a PTT (pulse transit time) feature; however, in practice, it cannot meet a goal accuracy when performing the blood pressure measurement.

Therefore, how to improve the accuracy of the blood pressure measurement has become a topic in the field.

### SUMMARY OF THE INVENTION

It is therefore an objective of the present invention to provide an image blood pressure measuring device and method using image pulse wave time difference, to improve an accuracy of the blood pressure measuring.

The present invention discloses a method for evaluating systolic and diastolic blood pressures of subject, performed by a processing module coupled to an image capturing module, wherein the image capturing module continuously records a face and a hand of a subject to continuously obtain multiple images of the hand and the face. The method includes, by the processing module, obtaining a biological information related to blood pressure of the subject according to the multiple images of the hand and the face captured by the image capturing module; and by the processing module, obtaining prediction results of systolic and diastolic blood pressures of the subject according to the biological information related to blood pressure of the subject.

A purpose of the present invention is to: obtain the biological information related to blood pressure of the subject and pulse wave time difference signal of the hand and the face of the subject according to images captured by the image capturing module, and obtain a PTT signal feature according to the biological information related to blood pressure and the pulse wave time difference signal, so as to predict systolic and diastolic blood pressures of the subject according to the PTT signal feature.

These and other objectives of the present invention will no doubt become obvious to those of ordinary skill in the art after reading the following detailed description of the preferred embodiment that is illustrated in the various figures and drawings.

### BRIEF DESCRIPTION OF THE DRAWINGS

FIG. 1 is a functional block diagram of a measuring system according to an embodiment of the present invention.

FIG. 2 is a flowchart of obtaining hand and face pulse signal feature according to an embodiment of the present invention.

FIG. 3 is a flowchart according to an embodiment of the present invention.

FIG. 4 is a flowchart according to an embodiment of the present invention.

FIG. 5 is a schematic diagram of face BMI feature calculation according to an embodiment of the present invention.

### DETAILED DESCRIPTION

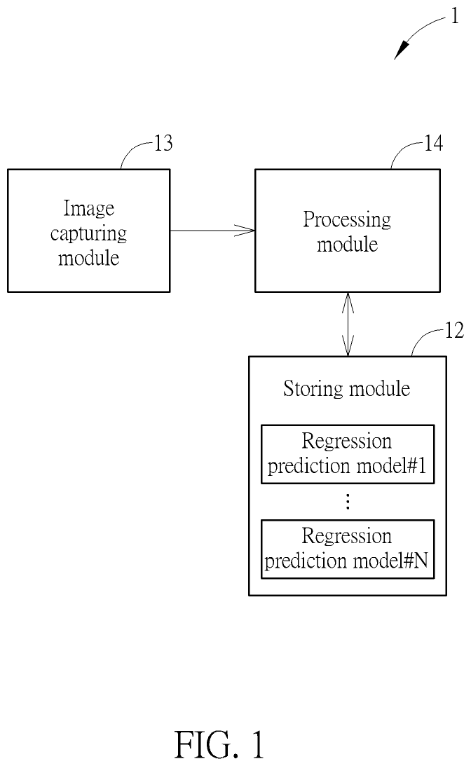

Please refer to FIG. 1, which is a functional block diagram of a blood pressure measuring system 1 according to an embodiment of the present invention. The blood pressure measuring system 1 is configured to execute a method of evaluating systolic and diastolic blood pressures of a subject, and includes a processing module 14, an image capturing module 13, and a storing module 12. The processing module 14 is coupled to the image capturing module 13 and the storing module 12, and configured to processing images outputted by the image capturing module 13.

The storing module 12 is configured to store given learning sampling features, e.g., multiple regression prediction model #1 to regression prediction model # N trained by KNN (k-nearest neighbors) learning method or ANN (artificial neural network) algorithm, which is not limited. The regression prediction model includes a BMI (body mass index) prediction model and a systolic and diastolic blood pressures measuring model. In this embodiment, the storing module 12 may be a hard drive or a memory device, which is not limited.

The image capturing module 13 is configured to continuously record a subject (e.g., continuously recording 45 seconds), to continuously obtain multiple color images associated with the subject. In this embodiment, the image capturing module 13 may be a camera with a frame rate of 90 frame/second, which is not limited.

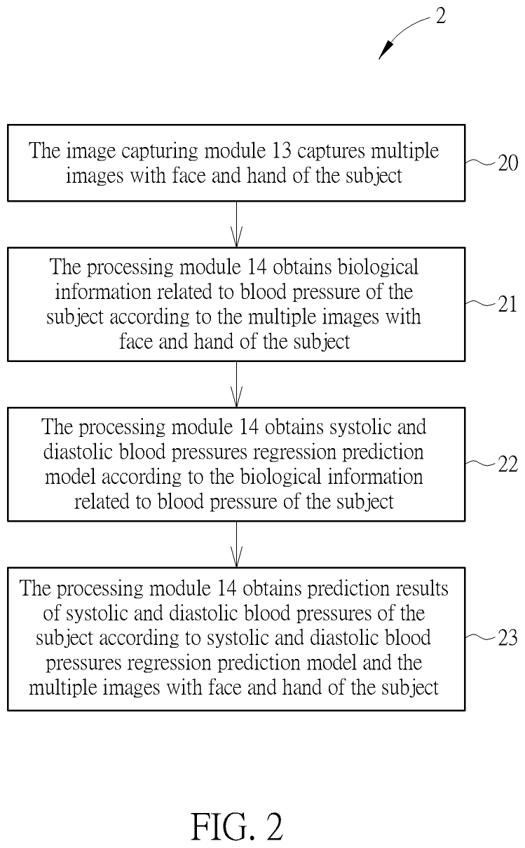

Please refer to FIG. 2, which is a flowchart of a process 2 of measuring systolic and diastolic blood pressures according to an embodiment of the present invention. The process 2 includes the following steps.

Step 20: The image capturing module 13 captures multiple images with face and hand of the subject.

Step 21: The processing module 14 obtains biological information related to blood pressure of the subject according to the multiple images with face and hand of the subject.

Step 22: The processing module 14 obtains systolic and diastolic blood pressures regression prediction model according to the biological information related to blood pressure of the subject.

Step 23: The processing module 14 obtains prediction results of systolic and diastolic blood pressures of the subject according to systolic and diastolic blood pressures regression prediction model and the multiple images with face and hand of the subject.

In Step 20, the multiple images captured by the image capturing module 13 include the face and the hand of the subject, and the multiple images are outputted to the processing module 14. In one embodiment, the image capturing module 13 is configured to capture scattered lights of the face and the hand of the subject.

In step 21, for each image, the processing module 14 respectively captures a face area image and a hand area image of the subject from each image to obtain the biological information related to blood pressure of the subject. In one embodiment, the processing module 14 may utilize machine learning to recognize the face area image and the hand area image of the subject from each image, and then convert corresponding rPPG (Remote PhotoPlethysmoGraphy) into pulse signals of the face and the hand. In one embodiment, the processing module 14 may obtain the biological information related to blood pressure of the subject according to continuous face rPPG and hand rPPG, wherein the biological information related to blood pressure includes at least one of a PTT (Pulse transit time), a BMI (Body mass index) feature, a heart rate, a pulse signal, and a blood oxygen value.

In step 22, the processing module 14 obtains the systolic and diastolic blood pressures regression prediction model according to the biological information related to blood pressure of the subject. In one embodiment, the systolic and diastolic blood pressures regression prediction model may be constructed according to at least one of a BMI feature, a fat index, a hand pulse wave signal, a face pulse wave signal, and a hand and face pulse wave time difference signal.

In step 23, the processing module 14 obtains the biological information related to blood pressure according to multiple images with the face and the hand of the subject, and utilizes time domain features such as the pulse wave signal to perform KNN or ANN algorithm to obtain the prediction results of the systolic and diastolic blood pressures of the subject. In particular, in this embodiment, the processing module 14 may obtain the prediction results of the systolic and diastolic blood pressures only according to the biological information related to blood pressure, and utilize the trained systolic and diastolic blood pressures regression prediction model. In particular, when the systolic and diastolic blood pressures regression prediction model obtains the prediction results only according to the biological information related to blood pressure, which means that the blood pressure regression prediction model is trained by a regression prediction algorithm (e.g., KNN and ANN) in cooperation with training data corresponding to the biological information related to blood pressure, which is not limited. In particular, when the prediction results of blood pressure are obtained by the biological information related to blood pressure, which means that the blood pressure regression prediction model is trained by a regression algorithm (e.g., KNN or ANN) in cooperation with training data of corresponding biological information related to blood pressure and the BMI feature of the subject, which is not limited to KNN or ANN. In particular, the processing module 14 may control the storing module 12 to store the biological information related to blood pressure and a feature of a pulse wave tome domain time difference signal to increase feature's database, so the regression prediction model may utilize the database for analysis.

Take the database combined with a machine learning model for example, the blood pressure measuring system 1 may utilize a sphygmomanometer certificated by United States FDA (Food and Drug Administration) to measure practical blood pressure, and then utilize the image capturing module 13 to continuously capture multiple images of the subject (e.g., capturing image for 45 seconds), the processing module 14 may utilize KNN or ANN to calculate a feature of a pulse wave time difference of the face and the hand of the subject, so as to construct a database by the practical blood pressure and the corresponding feature. When performing machine learning, the processing module 14 may utilize KNN or ANN to calculate time domain biological information associated with blood pressure of the subject (e.g., a feature of pulse wave time difference of the face and the hand), perform prediction according to the obtained time domain biological information and the feature database, and then calculate an average among blood pressure measuring results to be a final prediction result of blood pressure.

Take KNN algorithm for example, the processing module 14 may utilize a series of computations to calculate a feature of pulse wave time difference of the face and the hand of the subject, and utilize KNN algorithm to determine a K value to obtain blood pressure values corresponding to K pieces of data that are nearest to the feature of pulse wave time difference, and take an average among the K blood pressure values to obtain the prediction result of blood pressure.

In one embodiment, in Steps 21 and 23, the processing module 14 generates and transmits an notification message to image capturing module 13 to notify the subject to move his or her hand to a filming range for blood pressure measurement.

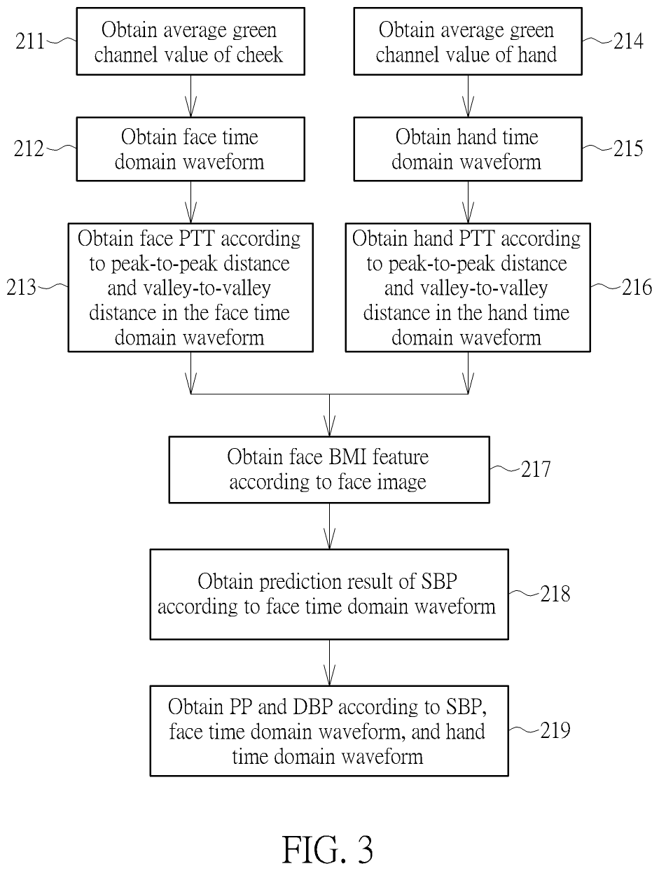

In particular, as shown in FIG. 3, step 21 further includes sub-step 211, 212, 213, 214, 215, 216, 217, 218, and 219.

Sub-steps 211 to 213 are configured to obtain a face time domain waveform and a face PTT. In sub-step 211, for each image, the processing module 14 obtains an average green channel value of a cheek of the subject in the image. In particular, in this embodiment, the processing module 14 firstly converts all green channel values from a raw image, and then calculates the average green channel value among the green channel values of the cheek to obtain the average green channel value. Wherein, a green channel value of each pixel of the cheek is a normalized value among all the green image values, or the green channel value may be a composition pixels of multiple normalized color channel signals, e.g., R*0.299+G*0.587+B*0.114, wherein R is a red value, G is a green value, B is a blue value, which is not limited. RGB values of each pixel of the cheek may be adjusted according to practical requirements or image characteristics when using different color light images.

In sub-step 212, the processing module 14 obtains a face time domain waveform of the subject according to the average green channel value of the cheek of each image. In particular, face blood flow varies as the heartbeat varies, such face blood flow causes color change to the face, based on this principle, heartbeat pulse wave of the face of the subject may be obtained according to the variation of the average green channel value of the face of each image. In one embodiment, the processing module 14 obtains a face image rPPG signal according to multiple average green channel values of the face; and then calculates a time domain waveform of heartbeat pulse wave of the subject according to the face image rPPG signal.

In sub-step 213, the processing module 14 obtains a time domain biological information related to blood pressure (including but not limited to multiple pulse wave peaks, multiple pulse wave valleys, and PTT) according to a peak-to-peak distance and a valley-to-valley distance in the face time domain waveform. In particular, in sub-step 213, peak-to-peak and valley-to-valley distances are obtained after noises (such as small peaks, and pulse wave feature out of heartbeat frequency range) are eliminated.

Sub-steps 214 to 216 are configured to obtain a hand time domain waveform and a hand PTT. In sub-step 214, for each image, the processing module 14 obtains an average green channel value of a hand of the subject in the image. In sub-step 215, the processing module 14 obtain the hand time domain waveform related to the subject (i.e., a heartbeat pulse wave corresponding to the hand) according to the average green channel value of the hand of each image.

In sub-step 216, the processing module 14 obtains the PTT related to blood pressure in the biological information according to a peak-to-peak distance and a valley-to-valley distance in the hand time domain waveform. In particular, in step 216, peak-to-peak and valley-to-valley distances are obtained after noises (such as small peaks, and pulse wave feature out of heartbeat frequency range) are eliminated.

In sub-step 217, the processing module 14 calculates a face BMI feature according to face image to obtain a BMI feature of the biological information related to blood pressure. In particular, the subject may be underweight with a low BMI range (<18 kg/m\hat{ }2), normal weight with a normal BMI (18˜23 kg/m\hat{ }2), or overweight with a high BMI (23˜27 kg/m\hat{ }2), and fat with very high BMI (>28 kg/m\hat{ }2). In particular, the processing module 14 may utilize the face BMI feature and corresponding fat index (i.e., parameters for indicating BMI range corresponding to underweight, normal, overweight and fat) for blood pressure prediction.

In sub-step 218, the processing module 14 obtains a measuring result of a systolic blood pressure according to the face time domain waveform. In particular, in this embodiment, the processing module 14 obtains a PTT feature within a time interval according to the heartbeat time domain waveform, and then infers the measuring result of a SBP (Systolic blood pressure) according to the PTT feature.

In sub-step 219, the processing module 14 according to face and hand time domain waveform, obtain PP (pulse pressure) and DBP (diastolic blood pressure). In particular, in this embodiment, the processing module 14 obtains a PTT feature within a time interval according to the time domain waveform, and then infers the measuring result of the PP according to the PTT feature. By calculating a difference between the SBP and PP, the DBP can be obtained.

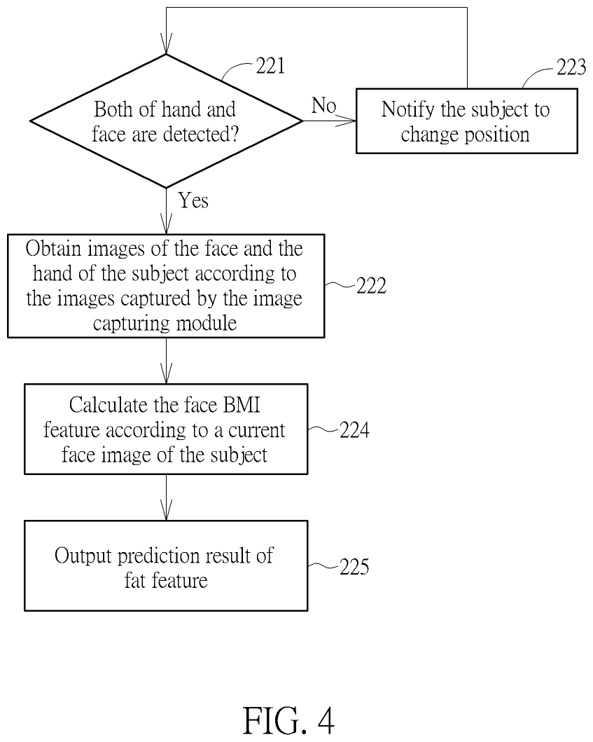

In particular, as shown in FIG. 4, step 22 further includes sub-steps 221, 222, 223, and 224.

In sub-step 221, the processing module 14 determines whether both the hand and the face of the subject are detected, and executes step 222 if yes, or executes step 223 if no. In step 223, the processing module 14 notifies the subject to change position to continue measurement, and return to step 221. In step 222, the processing module 14 obtains images of the face and the hand of the subject according to the images captured by the image capturing module. In sub-step 224, the processing module 14 calculates the face BMI feature according to a current face image of the subject, to output a fat feature of the biological information related to blood pressure of the subject. In sub-step 225, the processing module 14 outputs a prediction result of the fat feature for the following operations of the machine learning and ANN algorithm, which is not limited.

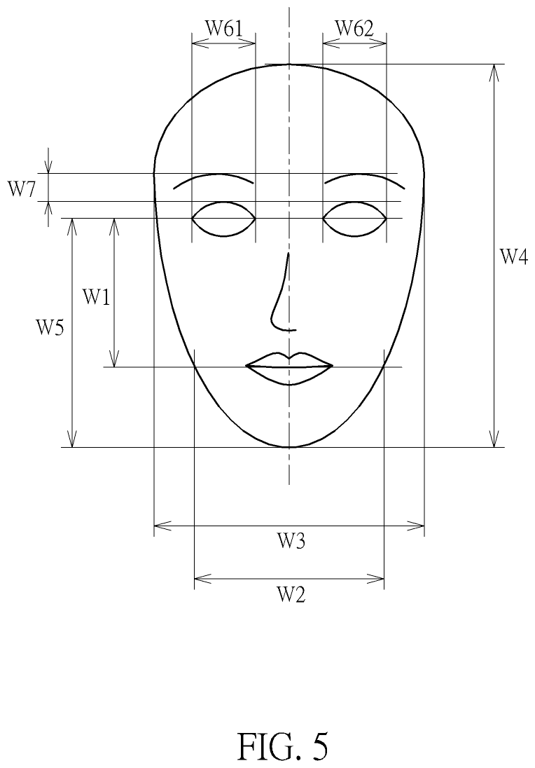

FIG. 5 is a schematic diagram of face BMI feature calculation according to an embodiment of the present invention. The face BMI feature includes but not limited to a ratio W1/W2, wherein W1 is a distance from a middle point of eyes to a center of lips and W2 is a face width corresponding lip's height; a ratio W3/W2, wherein W3 is a face width W3 corresponding to eye's height and W2 is a face width W2 corresponding lip's height; a ratio W5/W4, wherein W5 is a distance from the middle point of eyes to a center of a chin and W4 is a face height; an average width ((W61+W62)/2) of a right eye width W61 and a left eye width W62; and an eyelid height W7.

To sum up, the method of evaluating systolic and diastolic blood pressures of the subject of the present invention utilizes the processing module 14 to obtain biological information related to blood pressure and BMI feature according to the images captured by the capturing module 13, and utilizes the regression prediction model trained by ANN or KNN to perform blood pressure prediction, so as to the obtain prediction results of systolic and diastolic blood pressures of the subject. Therefore, the present invention may determine the blood pressure of the subject according to the prediction results.

Those skilled in the art will readily observe that numerous modifications and alterations of the device and method may be made while retaining the teachings of the invention. Accordingly, the above disclosure should be construed as limited only by the metes and bounds of the appended claims.

What is claimed is:

1. A method for evaluating systolic and diastolic blood pressures of subject, performed by a processing module coupled to an image capturing module, wherein the image capturing module continuously records a face and a hand of a subject to continuously obtain multiple images of the hand and the face, the method comprising:
by the processing module, obtaining a biological information related to blood pressure of the subject according to the multiple images of the hand and the face captured by the image capturing module; and
by the processing module, obtaining prediction results of systolic and diastolic blood pressures of the subject according to the biological information related to blood pressure of the subject.

2. The method for evaluating systolic and diastolic blood pressures of subject of claim 1, wherein before obtaining the prediction results of systolic and diastolic blood pressures of the subject according to the biological information related to blood pressure of the subject, further comprising:
by the processing module, obtaining a BMI (body mass index) of the subject according to one of the multiple images of the hand and the face; and
by the processing module, obtaining features required for a blood pressure regression prediction model of the subject according to the BMI of the subject.

3. The method for evaluating systolic and diastolic blood pressures of subject of claim 1, further comprising:
by the processing module, obtaining a blood pressure time domain biological information of the subject according to the multiple images of the hand and the face captured by the image capturing module; and
by the processing module, obtaining measuring results of the systolic blood pressure and the diastolic blood pressure of the subject according to the blood pressure time domain biological information.

4. The method for evaluating systolic and diastolic blood pressures of subject of claim 3, wherein, by the processing module, obtain the blood pressure time domain biological information of the subject according to the multiple images of the hand and the face captured by the image capturing module comprises:
for each one of the multiple images of the hand and the face, by the processing module, respectively obtaining a face average green channel value of the face of the subject and a hand average green channel value of the hand of the subject according to the multiple images of the hand and the face;
by the processing module, obtaining a time domain waveform related to a heartbeat pulse wave of the subject according to the face average green channel value and the hand average green channel value; and
by the processing module, obtaining the blood pressure time domain biological information according to multiple peak-to-peak distance in the time domain waveform, wherein the blood pressure time domain biological information comprises multiple pulse wave peaks, multiple pulse wave valleys, and multiple PTTs (pulse transit time) of the subject.

5. The method for evaluating systolic and diastolic blood pressures of subject of claim 4, wherein after for each one of the multiple images of the hand and the face, by the processing module, respectively obtaining the face average green channel value of the face of the subject and a hand average green channel value of the hand of the subject according to the multiple images of the hand and the face further comprising:
by the processing module, obtaining a face image rPPG (Remote PhotoPlethysmoGraphy) and a hand image rPPG according to the face average green channel value and the hand average green channel value; and
by the processing module, calculating a face time domain waveform and a hand time domain waveform related to the heartbeat pulse wave of the subject according to the face image rPPG and the hand image rPPG.

6. The method for evaluating systolic and diastolic blood pressures of subject of claim 3, wherein by the processing module, obtaining the measuring results of the systolic blood pressure and the diastolic blood pressure of the subject according to the blood pressure time domain biological information comprises:
by the processing module, obtaining the subject the measuring result of the systolic and diastolic blood pressures of the subject according to the blood pressure time domain biological information and a PTT of the subject.

7. The method for evaluating systolic and diastolic blood pressures of subject of claim 1, further comprising:
by the processing module, calculating multiple face BMI features of the subject according to a face area image from the multiple images of the hand and the face; and
by the processing module, obtaining the measuring result of the systolic and diastolic blood pressures of the subject according to the multiple face BMI features.

8. The method for evaluating systolic and diastolic blood pressures of subject of claim 7, wherein the multiple face BMI features comprise a first ratio of a distance from a middle point of eyes to a center of lips and a face width corresponding lip's height; a second ratio of a face width W3 corresponding to eye's height and a face width corresponding lip's height; a third ratio of a distance from the middle point of eyes to a center of a chin and a face height; an average width of a right eye width and a left eye width, and an eyelid height.

9. The method for evaluating systolic and diastolic blood pressures of subject of claim 1, further comprising:
by the processing module, when determining the multiple images of the hand and the face are not obtained, generating and transmitting a notification message to the image capturing module to notify the subject to move his or her hand and face to a filming range of the image capturing module.
